(19) United States
(12) Patent Application Publication
(10) Pub. No.: US 2017/0307595 A1
(43) Pub. Date: Oct. 26, 2017

(54) METHODS AND DEVICES TO STUDY METABOLISM

(71) Applicant: The General Hospital Corporation, Boston, MA (US)

(72) Inventors: Martin Yarmush, Newton, MA (US); Rohit Jindal, Braintree, MA (US); Shyam Sundhar Bale, Cambridge, MA (US); Osman Berk Usta, Watertown, MA (US)

(21) Appl. No.: 15/520,671

(22) PCT Filed: Oct. 21, 2015
(86) PCT No.: PCT/US15/56662

§ 371 Date: Apr. 20, 2017

Related U.S. Application Data
(60) Provisional application No. 62/067,239, filed on Oct. 22, 2014.

(51) Int. Cl. G01N 33/50

(52) U.S. Cl. CPC G01N 33/5067; G01N 33/502

(57) ABSTRACT

Methods and devices to screen test compounds, e.g., study metabolism of test compounds, e.g., a pro-drug, by one cell, e.g., a hepatocyte, and the effect of metabolism of the test compound by the first cell on a second cell, e.g., a cancer cell, are described.

### CROSS-REFERENCE TO RELATED APPLICATIONS

This application claims the benefit of U.S. application No. 62/067,239, filed on Oct. 22, 2014, which is incorporated herein by reference in its entirety.

### FEDERALLY SPONSORED RESEARCH OR DEVELOPMENT

This invention was made with U.S. Government support under Grant Number 1UH2TR00503-01 awarded by the National Institutes of Health. The Government has certain rights in the invention.

### TECHNICAL FIELD

This invention relates to methods and devices to study metabolism of test compounds, e.g., pro-drugs, by one type of cell, e.g., a hepatocyte, and the effect of metabolism of the test compound on a second type of cell, e.g., a cancer cell.

### BACKGROUND

The liver is the primary organ where drugs are metabolized. While drug-induced liver injury remains an important reason for withdrawal of therapeutic drugs, metabolites generated by the liver can also be responsible for toxicity in other organs and tissues (Kaplowitz, Drug-Induced Liver Injury: Introduction and Overview, in Drug Induced Liver Disease-Third Edition, Kaplowitz and DeLeve, Eds., Elsevier, 2013; and Schuster et al., Curr Pharm Des 11(27):3545-59, 2005). Progress has been made in the development of platforms for evaluating drug hepatotoxicity, including incorporation of hepatic cell lines or primary hepatocytes in various monoculture and co-culture configurations (Bale et al., Exp Biol Med, 2014; Godoy et al., Arch Toxicol 87(8):1315-1530, 2013; Dash et al., Expert Opinion on Drug Metabolism & Toxicology 5:1159-74, 2009). Nonetheless, development of systems that facilitate evaluation of drug toxicity in other organs or tissues as a result of metabolite(s) generated by the liver has lagged.

Systems presently used typically include interconnected chambers in which each chamber is seeded with a tissue or organ model and communication is achieved by flowing fluid across and between the chambers (Sung et al., Lab on a Chip 9:1385-94, 2009; Ma et al., Biomaterials 33(17):4353-61, 2012; Chao et al., Biochemical Pharmacology 78:625-632, 2009). In many cases, a “liver” chamber is seeded with a hepatocyte cell line (Sung et al., Lab on a Chip 9:1385-94, 2009; Ma et al., Biomaterials 33(17):4353-61, 2012), which generally lacks metabolic function in comparison to primary hepatocytes (Szabo et al., PloS ONE 8(3):e59432, 2013). While such models may provide an initial assessment for drug toxicity and multi-cell interactions, most of these in vitro models incorporate high media volumes (including tubing and reservoir volumes) creating an artificially high cell-media ratio, leading to loss of sensitivity in detecting product interactions, particularly in the case of unstable metabolites (Sung et al., Lab on a Chip 9:1385-94, 2009; Ma et al., Biomaterials 33(17):4353-61, 2012).

### SUMMARY

The present disclosure is based, at least in part, on the discovery that inter-tissue drug toxicity and metabolite effects can be determined using the two-chambered devices described herein in a simple static system without the need for any active flow of fluids. Accordingly, the present specification provides devices that include or consist of a solid substrate; a first member including a first inlet, a first chamber, and a first outlet; a second member including a second inlet, a second chamber, and a second outlet; and a liquid-permeable, cell-impermeable membrane; wherein the first member is fixed to the substrate, and wherein the membrane is sandwiched and secured between the first and second members to provide a liquid permeable, cell-impermeable barrier between the first and second chambers.

In some embodiments, the solid substrate is a glass slide. In certain embodiments, the first and second members include one or more of polydimethyl-siloxane (PDMS), polystyrene, and cyclic olefin copolymer (COC). In yet other embodiments, the membrane can be made of or include polyethylene terephthalate (PET), polycarbonate, nylon, Mylar, stainless steel, wire mesh, aluminum, synthetic mesh, spectra, Kevlar, plastic, or paper, and the membrane can be secured to the first and second members by, e.g., an adhesive or a liquid PDMS solution that can be polymerized in place.

In another aspect, the present disclosure provides methods of screening, e.g., determining the metabolism of, a test compound. The methods include or consist of: (a) providing one or more of the devices described herein; (b) introducing a suspension of first cells and a test compound into the first chamber of the device through the first inlet; (c) introducing a suspension of second cells into the second chamber of the device through the second inlet; (d) culturing the first and second cells by incubating the device; and (e) determining viability of the second cells in the second chamber, thereby determining the metabolism of the test compound by the first cells.

In some embodiments, the first cells are hepatocytes, e.g., rat hepatocytes. In some embodiments, the second cells are cancer cells, e.g., breast cancer cells, primary cells, or renal proximal tubule cells. In some embodiments, the test compound is tegafur, 4-ipomeanol, dacarbazine, trofosfamide, ifosfamide, or cyclophosphamide. In one embodiment, the device is incubated at 37° C. at about 5% CO₂, e.g., about 6% CO₂, 7% CO₂, 8% CO₂, 9% CO₂, or about 10% CO₂. In some embodiments, between 0.5 microliters and 20 microliters of the suspension of first cells is introduced into the first chamber of the device, e.g., 1 microliter, 1.5 microliters, 2 microliters, 2.5 microliters, 3 microliters, 4 microliters, 5 microliters, 6 microliters, 8 microliters, 10 microliters, 12 microliters, 14 microliters, 16 microliters, or 18 microliters. In some embodiments, between 0.5 microliters and 20 microliters of the suspension of second cells is introduced into the second chamber of the device, e.g., 1 microliter, 1.5 microliters, 2 microliters, 2.5 microliters, 3 microliters, 4 microliters, 5 microliters, 6 microliters, 8 microliters, 10 microliters, 12 microliters, 14 microliters, 16 microliters, or 18 microliters. In yet other embodiments, a ratio of volume of liquid in a chamber to the number of cells in the chamber ranges from 0.1 to 2.0 nanoliters per cell, e.g., 0.25. 0.5, 0.75, 1.0, 1.25, 1.5, 1.75, or 2.0 nanoliters per cell.

Unless otherwise defined, all technical terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control. Other features and advantages of the invention will be apparent from the following detailed description and figures, and from the claims.

### DESCRIPTION OF DRAWINGS

FIGS. 1A to 1E are a series of five schematic figures showing one embodiment of the assembly of a two-chamber microfluidic device as described herein. FIG. 1A shows the device components including a second (top) chamber (PDMS cast), a laser cut tissue-culture membrane, a first (bottom) chamber (PDMS sheet) and a substrate, e.g., a glass slide. The second (top) chamber has ports for access to both second (top) and first (bottom) chambers.

FIG. 1B is a diagram showing the tissue culture membrane attached to the second (top) chamber (spin-coated PDMS, 10 μm) and the first (bottom) chamber bonded by plasma treatment onto a glass slide.

FIG. 1C shows the second (top) chamber with membrane and first (bottom) chamber on a glass slide aligned and bonded using plasma treatment.

FIG. 1D shows a top view of the assembled two-chamber device with inlet and outlet ports for the second (top) chamber and first (bottom) chamber.

FIG. 1E shows a cross-section of the two-chamber device showing cancer cells, e.g., MCF-7 cells, seeded in the second (top) chamber (e.g., 100 μm thick) and normal, healthy cells, e.g., hepatocytes, seeded in the first (bottom) chamber (e.g., 250 μm thick).

FIGS. 2A and 2B are bar graphs comparing enzyme activity and product accumulation in a 12-well plate and a single chamber microfluidic device. Rate of product formation of Resorufin® (CYP1A1/2) (FIG. 2A) and B) Luciferin (CYP 3A4) (FIG. 2B) in both culture formats.

FIGS. 3A and 3B are a series of schematic figures showing tegafur metabolism and 5-fluorouracil (5-FU) toxicity comparison in a 12-well plate and a microfluidic device a described herein.

FIG. 3A is a figure showing metabolic conversion of tegafur to 5-FU by CYP present in hepatocytes. Tegafur is a non-toxic pro-drug, whereas 5-FU is toxic to dividing, cancerous cells.

FIG. 3B shows cell placement within the two-chamber device. Hepatocytes in the lower chamber convert tegafur into 5-FU, which is taken up by MCF-7 cells in the second (top) chamber resulting in cell death and is measured by LDH release.

FIG. 3C is a bar graph comparing LDH release of hepatocyte, MCF-7, and co-culture exposure to 100 μM tegafur+100 μM uracil in a 12-well plate and a microfluidic device as described herein. Single cell controls were performed in single chamber devices.

FIGS. 4A and 4B are two bar graphs depicting mass spectrometry analysis of tegafur and uracil consumption, and 5-FU production in microfluidic devices described herein. Kinetics showing of tegafur, uracil consumption (FIG. 4A) and 5-FU production (FIG. 4B) in single chamber device seeded with rat primary hepatocytes.

FIGS. 5A and 5B are two bar graphs depicting mass spectrometry analysis of tegafur and uracil consumption, and 5-FU production in single chamber (control) and two-chamber (co-culture) microfluidic devices. FIG. 5A shows tegafur, and uracil consumption, and FIG. 5B shows 5-FU production after 24 hours of drug exposure (100 μM tegafur+100 μM uracil).

### DETAILED DESCRIPTION

Described herein is the fabrication and use of two-chambered devices for inter-tissue drug toxicity testing and for evaluating metabolite effects of test compounds. The microscale environment created in these devices enables cell culture in a low media-to-cell ratio leading to higher metabolite formation and rapid accumulation, which is lost in traditional plate cultures or other interconnected chamber models due to higher culture volumes. By building a two-chamber microfluidic device that allows for direct interaction through a permeable membrane, the need for any fluidic flow has been eliminated, creating a static system and greatly simplifying the model. These devices have been demonstrated for testing a chemotherapeutic pro-drug, tegafur-uracil, which when metabolized by primary hepatocytes, produces 5-fluorouracil (5-FU), a metabolite that is toxic to cancer cells. Conversion of the metabolite and its resultant toxicity are measureable in the microscale model within a few hours, e.g., 5, 10, 12, 15, 18, 20, 22, or 24 hours. Overall, the two-chamber devices provide a novel, easy-to-use platform for testing drug metabolism, toxicity, and interactions between multi-tissue systems.

In the context of drug metabolism, hepatocytes communicate with cells in other organs in the body via secreted metabolites. Traditionally, co-culture interactions mediated by secreted factors have been evaluated in transwell systems where different cell types are separated by a porous membrane that enables soluble factor communication. One limitation of these systems is the dilution of secreted factors due to exposure of the cells to high media volume (Wikswo, Experimental Biology and Medicine 239(9):1061-72, 2014; Mehling et al., Current Opinion in Biotechnology 25:95-102, 2014). These systems become especially limiting in settings where the secreted factor, such as a metabolite, is cleared by other mechanisms, preventing it from reaching toxic levels.

While known plate cultures have inherently high media-to-cell ratios, prior microfluidic interconnected chamber systems rely on fluidic connections and reservoirs, which eventually result in relatively high media-to-cell ratios. For drug screening and toxicity related studies, it is essential to develop in vitro models that incorporate both hepatocytes and target-organ cells of interest, while reducing the culture volume to increase metabolite production and interrogate resulting metabolite toxicity.

### Devices

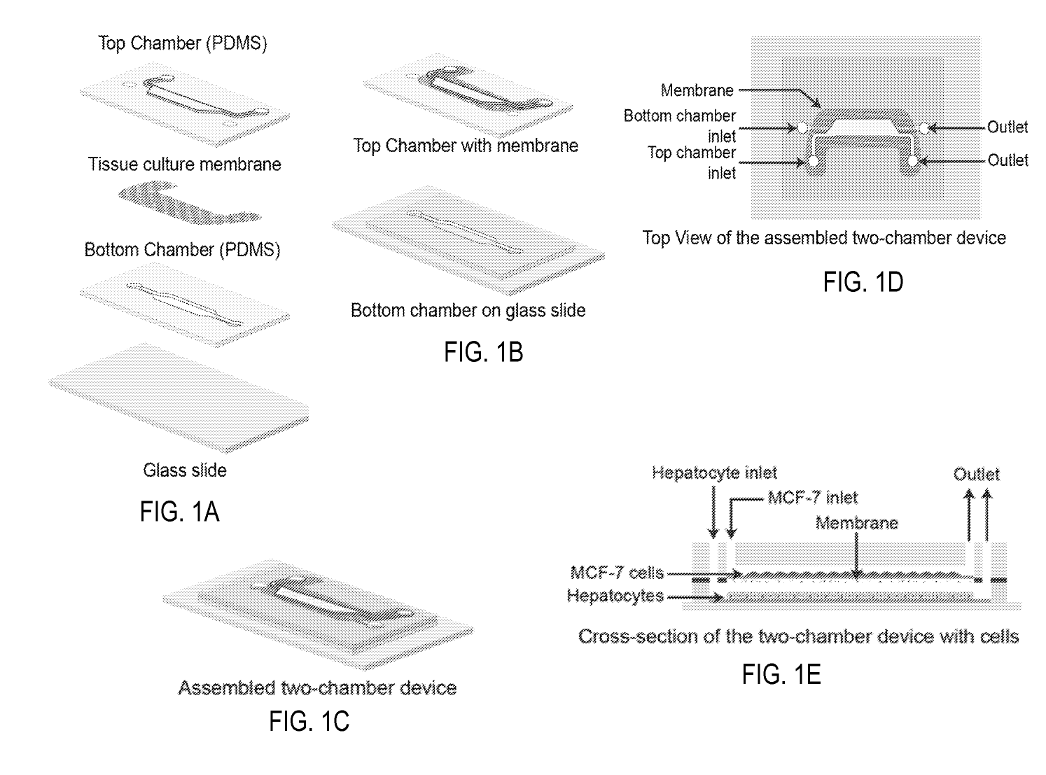

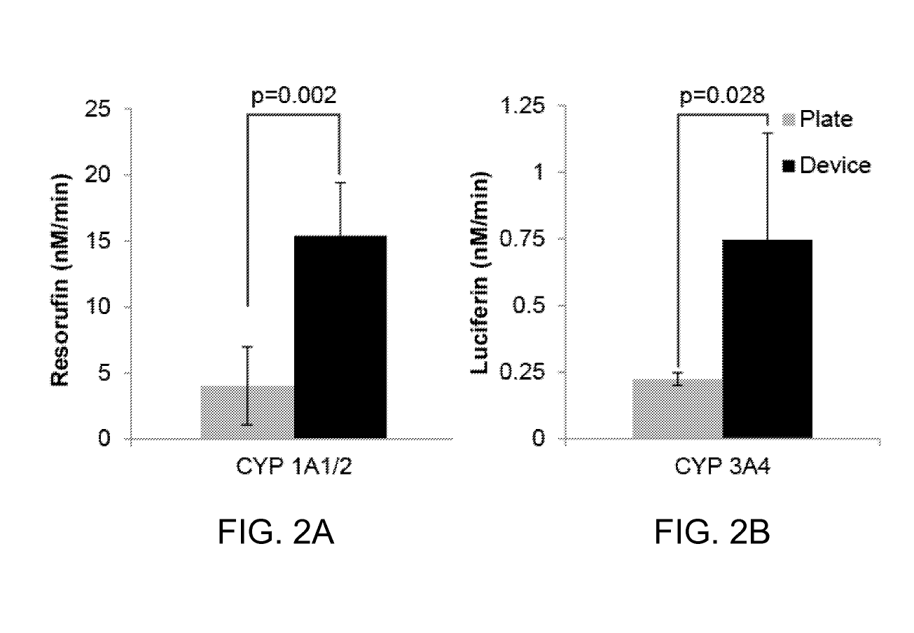

A microscale device with a two-chamber design separated by a semi-permeable, tissue-culture membrane was developed to enable the culture of two different cells within the same device, while addressing separate cell populations within each chamber (FIGS. 1A to 1E). The use of a two-chamber device allows for the culture of primary hepatocytes in collagen gel (in the first (bottom) chamber), while media circulates around cancer cells seeded onto the semi-permeable membrane that separates the first (bottom) chamber from the second (top) chamber.

The two-chamber device is fabricated with a) a second (top) chamber that can be manufactured of a plastic or other inert material, e.g., polydimethylsiloxane (PDMS), polystyrene, or cyclic olefin copolymer (COC), (and have a height of about, e.g., 100 μm, e.g., 50 to 150 μm), b) a semi-permeable, e.g., liquid-permeable, cell-impermeable tissue culture membrane, c) a first (bottom) chamber cut from an inert plastic or other materials such as a PDMS, polystyrene, or COC sheet (and have a height of about, e.g., 250 μm, e.g., 100 to 350 μm), and d) a solid substrate, e.g., a glass slide. The second (top) and first (bottom) chambers can accommodate approximately 5,000 to 10,000 cells or more each (cultured on tissue culture membrane and glass slide, respectively), which are seeded separately from ports for each chamber.

In addition, as a control, single chamber devices were prepared by bonding a PDMS chamber with similar dimensions of the second (top) chamber (100 μm thick, 10 mm² area, 10,000 cells) onto a glass slide. Media volumes and other pertinent parameter for the well and device configurations are given in Table 1.

TABLE 1
Culture Parameters of Device and Plate Set-Up

| | Single | Two-chamber | | | 12 well plate | |
|---|---|---|---|---|---|---|
| | chamber | device | | 12 well | with transwell | |
| | device | Top | Bottom | plate | Transwell | Well |
| Culture | 10 mm² | 10 mm² | 10 mm² | 3.8 cm² | 1.12 cm² | 3.8 cm² |
| Area | | | | | | |
| # Cells | 10,000 | 10,000 | 10,000 | 500,000 | 150,000 | 500,000 |
| Volume | 1 μL | 1 μL | 2.5 μL | 500 μL | 500 μL | 1000 μL |

| | | | | |
|---|---|---|---|---|
| Media | 0.1 nL | 0.35 nL | 1 nL | 3 nL |
| volume/ | | | | |
| Hepato- | | | | |
| cyte | | | | |

In general, the two-chamber devices described herein feature a solid substrate; a first member comprising a first inlet, a first chamber, and a first outlet; a second member comprising a second inlet, a second chamber, and a second outlet; and a liquid-permeable, cell-impermeable membrane; wherein the first member is fixed to the substrate, and wherein the membrane is sandwiched and secured between the first and second members to provide a liquid-permeable, cell-impermeable barrier between the first and second chambers. The devices are further described below and represented in FIGS. 1A to 1E.

The liquid-permeable, cell-impermeable membrane can be constructed from any art-known filter material, e.g., PET, polycarbonate, nylon, Mylar, stainless steel, wire mesh, aluminum, synthetic mesh, spectra, Kevlar®, plastic, or paper. The liquid-permeable, cell-impermeable membrane can have a pore-size of about 0.2 μm to about 50 μm, e.g., about 0.22 μm, 0.4 μm, 0.5 μm, 0.8 μm, 1 μm, 1.2 μm, 1.5 μm, 2 μm, 2.2 μm, 2.5 μm, 3 μm, 4 μm, 5 μm, 6 μm, 10 μm, 12 μm, 14 μm, 16 μm, 18 μm, 20 μm, 22 μm, 25 μm, 28 μm, 30 μm, 33 μm, 35 μm, 38 μm, 40 μm, 42 μm, 45 μm, or about 48 μm. The cell culture surface, which is arranged at the bottom of the second (top) chamber, is derived from a standard tissue culture transwell membrane, and can be treated with extracellular matrix components (ECM) such as fibronectin, collagen, and matrigel using established protocols for favorable cell attachment. In some embodiments, the device is sterile. Skilled practitioners will appreciate that the liquid-permeable, cell-impermeable membrane can be secured within the body using any art-known method, e.g., using a liquid PDMS formulation, various adhesives, and/or by pressure.

### Methods of Determining Metabolism of a Test Compound

Described herein is a hepatocytecancer co-culture model by culturing hepatocytes (liver) and cancer cells (MCF-7, breast cancer) in the first (bottom) and second (top) chambers of the two-chamber devices, respectively. The metabolic conversion of tegafur, a pro-drug that is converted to 5-FU by hepatocytes, and its incorporation by dividing tumor cells (MCF-7), leads to their cell death. While the methods described herein utilize hepatocytes and cancer cells to study the metabolism of tegafur, the co-culture microfabricated devices can be adapted to interrogate metabolite-mediated toxicity using a variety of cells in multiple configurations. For example, in the first (bottom) chamber, rat hepatocytes, human hepatocytes, e.g., cryopreserved human hepatocytes, and hepatocyte-like cells, such as induced pluripotent stem cell-derived hepatic cells, can be cultured. The second (top) chamber has a tissue culture treated transwell membrane that is amenable to culture a variety of cells, including cell-lines (cancer model), primary cells, and other derived cells to generate a plethora of hepatocyte-cancer or hepatocyte-organ models.

Some examples of co-culture models include the second (top) chamber populated with primary renal proximal tubule cells to create a hepatocyte-kidney model for testing nephrotoxic metabolites, e.g., ifosfamide conversion to chloroacetaldehyde. Species-related toxicity can also be evaluated. For instance, efavirenz, an anti-retroviral drug for HIV treatment shows toxicity in rat models, unlike in human models. The present co-culture model can be adapted to culture A) rat hepatocytes rat primary renal proximal tubule cells and B) human hepatocytes human primary renal proximal tubule cells to compare toxic effects of metabolites.

### Test Compounds

Tegafur-uracil is a pro-drug that is widely used in chemotherapeutic applications for colorectal and breast cancer (Longley et al., Nature Reviews, Cancer 3(5):330-8, 2003). Briefly, orally administered tegafur is metabolized in the liver to form 5-Fluorouracil (5-FU), which gets incorporated into fast-dividing cells and cancerous cells. 5-FU is an analogue of uracil, which is an essential component during cell division and integrates into cellular DNA, inhibiting cell division (Longley et al., Nature Reviews, Cancer 3(5):330-8, 2003). However, 5-FU in the human body is degraded by a) conversion into secondary and tertiary metabolites and b) degradation by dihydropyrimidine dehydrogenase (DPD) (Longley et al., Nature Reviews, Cancer 3(5):330-8, 2003; Meropol et al., Cancer Chemotherapy and Pharmacology 43(3):221-6, 1999). Due to these processes, the half-life of 5-FU in circulation in the human body is very short ( 30 minutes), while DPD activity clears ˜80% of 5-FU produced (Longley et al., Nature Reviews, Cancer 3(5):330-8, 2003; Boisdron-Celle et al., Cancer Letters 249(2):271-82, 2007). Uracil is added in combination with tegafur to reduce pyrimidine catabolism and increase the longevity of 5-FU circulation. The low bioavailability of 5-FU and faster clearance makes understanding tegafur metabolism in vitro very challenging.

A variety of pro-drugs, especially the class of Cytochrome P450 activated pro-drugs, can be easily incorporated into these systems for testing purposes. Pro-drugs such as, but not limited to, 4-ipomeanol, dacarbazine, trofosfamide, ifosfamide, and cyclophosphamide can be tested. Further, any relevant pro-drug which can be converted by hepatocytes using other enzyme dependent pathways can be interrogated using this system.

### EXAMPLES

Several general protocols are described below, which can be used in any of the methods described herein and do not limit the scope of the invention described in the claims.

### Example 1

### Microfluidic Device Fabrication and Cell Culture

A two-chamber, membrane-based microfluidic device was fabricated at Massachusetts General Hospital's BioMEMS Research facility and assembled in the lab. Briefly, silicon-wafer templates served as negative molds to generate the top layer of the device in polydimethylsiloxane (PDMS, Sylgard 184, Dow Corning), using standard soft-lithography protocols (McDonald et al., Analytical Chemistry 74(7):1537-45, 2002). Using appropriate dimensions, a channel for the first (bottom) layer was laser-cut on a thin PDMS sheet (250 μm, HT-6240-.010, Rogers Corporation) and bonded to a 50×22 mm glass slide using oxygen plasma treatment followed by incubation at 70° C. for 10 minutes.

A liquid-permeable, cell-impermeable membrane such as a 3.0 μm pore sized polyethylene terephthalate (PET)-based transwell membrane insert (FisherSci, Cat No. 07-200-171) was cut to dimensions using a laser cutter.

The second (top) chamber of the device was first bonded to the membrane. Briefly, a 10 μm layer of PDMS pre-polymer was spin-coated onto a clean glass coverslip and a clean top layer was placed onto it for the PDMS to spread on the surface around the channel. A clean laser-cut membrane was then applied to the PDMS pre-polymer coated surface and bonded carefully while ensuring the channels remained free of PDMS pre-polymer and covered the ports for the second (top) chamber only. The second (top) layer with the membrane was cured at room temperature for 48 hours until the PDMS cured and held the membrane tightly. Once the second (top) and first (bottom) layers are assembled individually, they were then bonded to each other using oxygen plasma treatment, with the membrane being in the center of the device.

The assembled final device is then heated at 70° C. overnight to strengthen the bonds and stored in a dry, dark place until use.

### Example 2

### Co-Culturing Cells and Testing Compounds

Rat primary hepatocytes and human breast cancer cells (MCF-7) were co-cultured in the device of Example 1 to create a hepatocyte-cancer model for drug screening. In the two-chamber device, intercellular communication between hepatocytes and cancer cells is achieved without any flow within the devices. In the two-chamber device, both cell types were independently seeded into their respective chambers in the device using inlet ports on fibronectin-coated devices. Rat primary hepatocytes and MCF-7 cells were seeded into the first (bottom) and second (top) chambers, respectively. Control monocultures were seeded in a single chamber device. The co-culture model created provides a microenvironment with cells cultured in close proximity and relatively small volume. Co-culture of hepatocytes and MCF-7 cells was compared in 12-well plates against a two-chamber device. A two-chamber device co-culture requires 0.35 nL/hepatocyte volume, while similar culture in a 12 well plate requires 3 nL/hepatocyte (see Table 1).

Materials

Fibronectin (Cat No. F1141), Thiazolyl Blue Tetrazolium Bromide (MTT reagent, Cat No. M5655), Tegafur (Cat No. T7205), 5-fluorouracil (5-FU, Cat No. F6627) and Uracil (Cat No. U1128) were purchased from Sigma. DMEM (Cat No. 31600083), 0.05% Trypsin-EDTA (Cat No. 25300062), Williams E media (Cat No. A1217601), Epidermal Growth Factor (EGF, Cat No. E3476), Penicillin-Streptomycin (Cat No. 15140122), Glutamine (Cat No. 21051040) was purchased from Life Technologies. LDH assay kit was purchased from Promega (Cat No. G1780). Fetal Bovine Serum (FBS, Hyclone Cat No. SH30071.03), Glucagon (Bedford Laboratories, Cat No. 55390-004-01), Hydrocortisone (SOLU-CORTEF® hydrocortisone sodium succinate for injection, Pharmacia Corporation), Insulin (Eli Lily, Cat No. HI-213) were purchased and used as per manufacturer's directions. All other chemical reagents were purchased from Sigma.

Media Formulations

MCF-7 cell culture media was prepared with high glucose (4.5 g/L) DMEM supplemented with 10% FBS, 2mM Glutamine and 2% Penicillin-Streptomycin.

Hepatocyte maintenance media was prepared with high glucose (4.5 g/L) DMEM supplemented with 10% FBS, 20 μg/L EGF, 14.28 μg/L glucagon, 7.5 mg/L hydrocortisone, 500 U/L Insulin, 2 mM Glutamine and 2% Penicillin-Streptomycin.

Williams E medium was supplemented with, 20 μg/L EGF, 14.28 μg/L glucagon, 7.5 mg/L hydrocortisone, 0.05 U/L insulin, and 2% Penicillin-Streptomycin.

Rat Hepatocyte Isolation

Hepatocytes were obtained from female Lewis rat using two-step collagenase protocol. Two to three month old female Lewis rats (Charles River Laboratories, Wilmington, Mass.) weighing 180 to 200 g were used as a source of hepatocytes and were maintained in accordance with National Research Council guidelines. Experimental protocols were approved by the Subcommittee on Research Animal Care, Massachusetts General Hospital. Using a modification on the two-step collagenase perfusion method (Seglen, Methods Cell Biol 13:29-83, 1976; and Dunn et al., FASEB J3(2):174-7, 1989), which involves purification of cell suspension by means of centrifugation over percoll, we routinely isolated approximately 200 million hepatocytes per rat liver with viability between 85% and 98% as evaluated by Trypan blue exclusion.

MCF-7 Cell Culture

MCF-7 cells were maintained in DMEM at 37° C., 5% CO₂. Cells were grown to 80% confluency and trypsinized using Trypsin-EDTA and passaged at 1:10 dilution.

Toxicity Experiments

Toxicity experiments were performed in 96-well plates. Briefly, 96-well plates were coated with 50 μg/mL fibronectin for 1 hour at 37° C. Freshly isolated rat hepatocytes and MCF-7 cells were seeded at 50,000 cells/well in 100 μL of media and incubated overnight at 37° C., 10% CO₂. Hepatocytes were seeded in hepatocyte maintenance media while MCF-7 cells were seeded in DMEM. Media was replaced and cells were exposed to tegafur or 5-FU+Uracil in Williams E media at 37° C., 10% CO₂. Uracil concentration was maintained at 100 μM, while 5-FU concentration varied. After 24 hours of exposure, media were removed and cells were incubated with 0.5 mg/mL MTT reagent for 2 hours. Media were removed from the wells and 100 μL DMSO was added to each well and mixed on a shaker for 10 minutes. The absorbance was measured at 570 nm and IC-50 values were obtained using Sigmaplot software with a sigmoidal 4-parameter fit.

Seeding Cells into the Microfluidic Device Culture

Microfluidic devices were wiped clean with 70% isopropanol and sterilized under UV in a hood for 20-30 minutes. Both the second (top) and first (bottom) chambers of the device are then filled with 50 μg/mL fibronectin and incubated for at least 1 hour at 37° C. In the first (bottom) chamber, 10 μL of primary rat hepatocytes (5 million/mL), and in the second (top) chamber 10 μL of MCF-7 (10 million/mL) were introduced and incubated at 37° C., 10% CO₂ overnight. Media in the device was replaced with Williams E media for toxicity experiments.

Control Tests in a Transwell Culture System

Transwell experiments were performed in 12-well transwell culture systems with a 3.0 μm pore size. Briefly, well and transwell were coated with 50 μg/mL fibronectin and incubated for 1 hour at 37° C. To the well, 0.5 M freshly isolated rat hepatocytes were added and to the transwell, 0.15 M MCF-7 cells were added and incubated overnight at 37° C., 10% CO₂. Media was replaced with Williams E media for toxicity experiments.

CYP450 Assay

CYP450 1A1/2 activity was evaluated using 7-ethoxyresorufin. For hepatocytes in wells, 500 μL of substrate (10 μM 7-ethoxyresorufin+80 μM Dicumarol) in Williams E media was added and incubated at 37° C. 100 μL of the reagent was withdrawn at 15, 30, 45, and 60 minute intervals. For hepatocytes in devices, multiple devices (n=2 per time point) were used and reagent was collected at 15, 30, 45, and 60 minute intervals in 20 μL Williams E media, and diluted to 100 μL. Rate of resorufin production was calculated by diluting resorufin standard in Williams E media. Fluorescence from the collected sample was measured at λₑₓ=525±10 nm and λₑₘ=580±10 nm.

CYP450 3A4 activity was evaluated using CYP3A4 kit from Promega (Cat No. V9001) with setup similar to CYP450 1A1/2 assay. Hepatocytes in both wells and transwells were exposed to substrate solution (3 μM Luciferin-IPA) and media collected at 15, 30, 45, and 60 minute intervals. Media in the devices was collected in 20 μL Williams E media, and diluted to 100 μL. To 50 μL sample, 50 μL detection reagent was added and luminescence from the sample was measured with a 1 second integration time. Rate of luciferin production in the samples was calculated using beetle luciferin (Promega, Cat No. E1601).

Data Normalization and Statistics

Concentration of product formed in microfluidic devices was normalized to 1 μL of culture media within the device. Data was averaged from n=2 experiments with n=2 samples per experiment.

Lactose Dehydrogenase (LDH) Assay

LDH in the supernatant was evaluated using CytoTox 96 Non-Radioactive Cytotoxicity Assay (Promega Cat No. G1780). For plate assays, 50 μL of media was mixed with 50 μL reagent and incubated for 30 minute at room temperature in dark. For microfluidic devices, media in the device was collected in 20 μL of fresh Williams E media. To 5 μL of the media, 45 μL of Williams E media was added and mixed with 50 μL reagent and incubated for 30 minutes at room temperature in dark.

At the end of incubation, 50 μL stop solution was added and absorbance measured at 490 nm.

Data Normalization and Statistics

LDH from culture supernatant is normalized as LDH from cultures exposed to 100 μM tegafur+100 μM uracil to LDH from cultures exposed to vehicle control. Data and is averaged from n=2 experiments for plate with n=3 samples per experiment, and n=5 experiments for devices with n=2 or 3 per experiment. Data is plotted as fold change over controls. Percentage cell death was calculated as LDH in media/LDH in 10,000 MCF-7 cells.

Mass Spectrometry

Tegafur, 5-FU, and Uracil concentrations in media were quantified using an LC/MS-MS 3200 QTRAP Hybrid Triple Quadrupole Linear Ion Trap mass spectrometer (AB SCIEX, Foster City, Calif.) coupled to a 1200 Series Binary LC System (Agilent Technologies, Santa Clara, Calif.). A standard solution of each analyte was directly infused into the mass spectrometer, which was operating under negative mode with the following settings: Curtain gas (CUR) at 30.0, collision gas (CAD) at 5, IonSpray Voltage (IS) at −4500.0, the temperature (TEM) of the turbo gas in the TurbolonSpray at 400° C. and both Ion Source Gases (GS1 and GS2) at 60.0. Using Analyst software (Version 1.5, AB Sciex), a ‘Compound Optimization’ routine was performed to identify multiple reaction monitoring (MRM) precursor/product ion transition pairs for each analyte that maximizes peak intensity. The tunable MS-specific parameters for the optimization were the declustering potential (DP), the entrance potential (EP), the collision energy (CE), as well as the collision cell exit potential (CXP). The optimized parameters for each MRM transition are summarized in Table 2.

TABLE 2
MS-Specific Parameters

| | Precursor | Product | DP | EP | CE | CXP |
|---|---|---|---|---|---|---|
| Compound | (Da) | (Da) | (V) | (V) | (V) | (V) |
| Tegafur | 198.960 | 42.000 | −25 | −8 | −44 | −4 |
| 5-FU | 128.921 | 42.000 | −65 | −4.5 | −128 | −4 |
| Uracil | 110.890 | 42.000 | −55 | −9 | −130 | −2 |

MS data acquisition for each sample was achieved by injecting 10 μL, of media through a liquid chromatographic (LC) separation phase followed by simultaneous detection of all three MRM transitions, each with a dwell time of 500 ms. The LC method utilized a Synergy Hydro-RP (reverse phase) column (150 mm×2 mm inner 4 μm 80 Å particles; Phenomenex, Torrance, Calif.), which was kept at ambient temperature. The aqueous mobile phase A was HPLC grade water with 0.1% formic acid and the organic phase B was HPLC grade methanol with 0.1% formic acid. The elution gradient was set as: 0 minutes-3% B, 3 minutes-3% B, 12 minutes-95% B, 15 minutes-95% B, 18 minutes-3% B, and 25 minutes-3% B.

Results

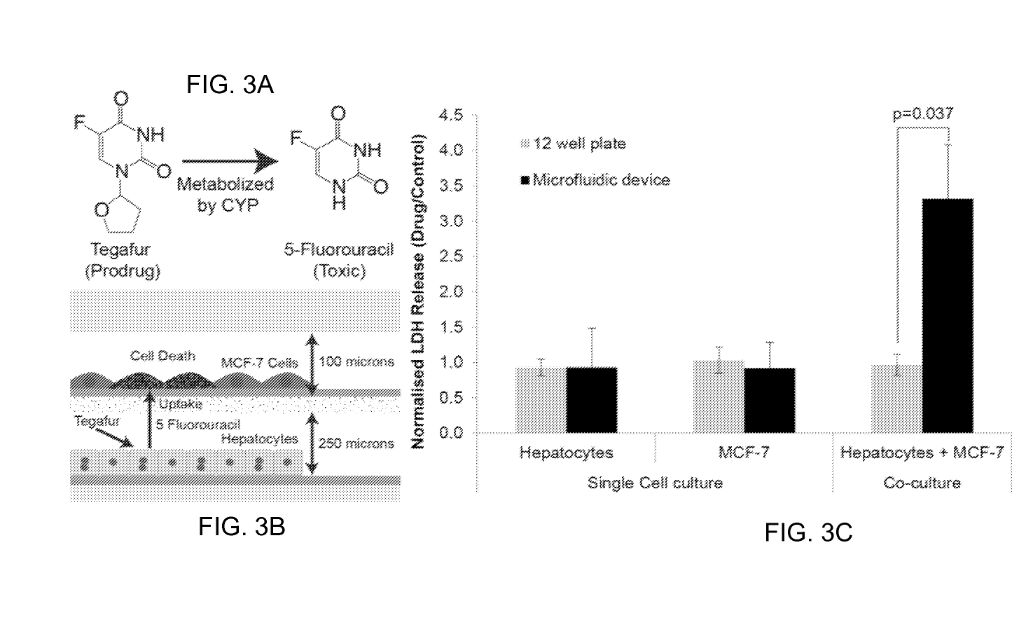

Metabolism of tegafur to 5-FU and the resulting toxicity was tested in both 12-well transwell and two-chamber microfluidic device with co-cultures exposed to 100 μM tegafur+100 μM uracil (FIGS. 3A and 3B). The dose of 100 μM for tegafur was chosen based on the concentration that is achieved by oral uptake of tegafururacil (UFT), Cₘₐₓ=31.159 μM, Area Under Curve (AUC)=121 μM (Meropol et al., Cancer Chemotherapy and Pharmacology 43(3):221-6, 1999). Furthermore, IC-50 values (Table 3) indicate that at the concentration of 100 μM tegafur is not toxic to either hepatocytes or MCF-7 cells. Cells in both co-culture formats were incubated with tegafur-uracil for 24 hours and media was collected and evaluated for LDH content. Single cell controls were prepared with single chamber device or 12 well plate. LDH release from drug-exposed samples was normalized with respective configurations exposed to vehicle only controls. The results indicate that there is an increase in the LDH release (˜3.5 times) in the case of micro-fluidic co-cultures, while there was no such increase in case of plate cultures or single cell controls (FIG. 3C). In addition, cell death as a result of MCF-7 cell exposure to 5-FU produced by metabolic conversion of tegafur is calculated as 12.4±1.6%. This is a clear demonstration of the advantages of a microscale cell culture model, which overcomes the challenges (dilution effects) of traditional plate techniques.

TABLE 3
IC-50 values of tegafur and (5-Fluorouracil + uracil)

| Cell type | Tegafur | 5-Fluorouracil + uracil |
|---|---|---|
| Rat hepatocytes | >1200 μM | >200 μM |
| MCF-7 | 340 μM | 13 μM |

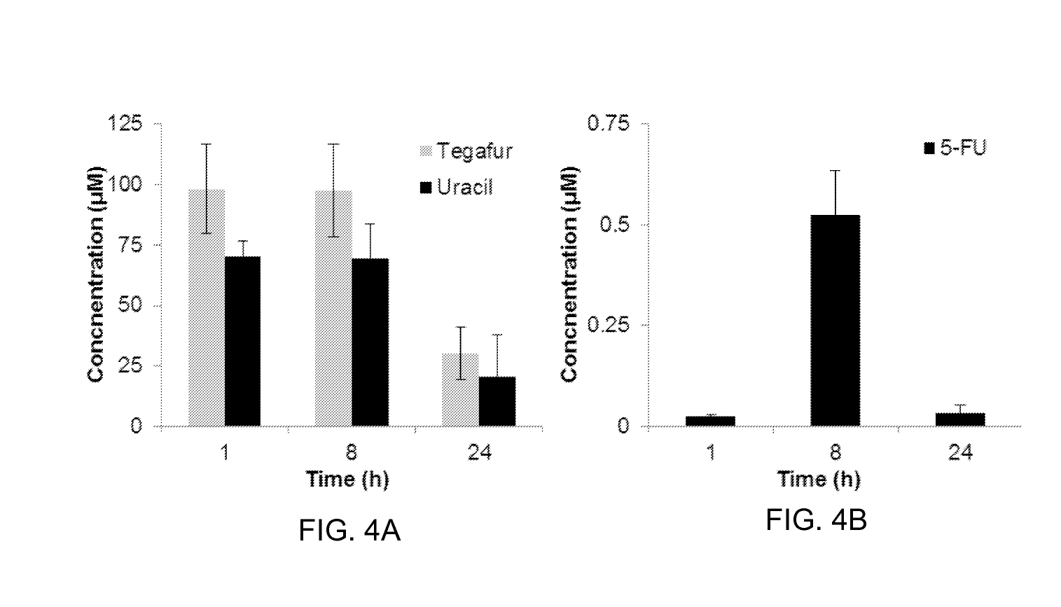

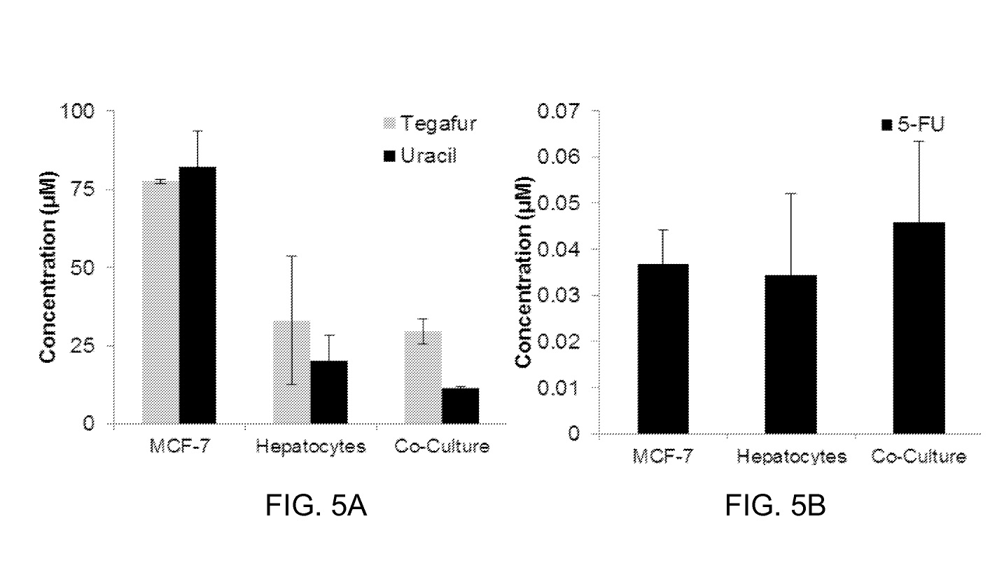

To further understand tegafur metabolism and kinetics, mass spectrometry was used to measure the concentrations of tegafur, uracil, and 5-FU within the media in the device. First, the kinetics of tegafur and uracil metabolism and 5-FU production was measured in a single chamber device with hepatocytes alone. Tegafur and uracil concentrations within the media shows a decrease ˜75% over a period of 24 hours and 5-FU shows an increase in production up to 8 hours and a subsequent decrease (FIGS. 4A and 4B). While tegafur and uracil are actively metabolized by hepatocytes, 5-FU is actively produced and subsequently converted by hepatocytes into secondary metabolites (Longley et al., Nature Reviews, Cancer 3(5):330-8, 2003). Further, the bioavailability of 5-FU is significantly lower (Cₘₐₓ=0.847 μM) when compared with tegafur (Cₘₐₓ=31.159 μM) due to its rapid clearance (Longley et al., Nature Reviews, Cancer 3(5):330-8, 2003; Meropol et al., Cancer Chemotherapy and Pharmacology 43(3):221-6, 1999). In co-culture drug exposure experiments within the two-chamber microfluidic device, measurements were made at the end of incubation (24 hours), and are shown in FIGS. 5A and 5B. A similar trend is noticed in these cultures, showing a decrease in tegafur and uracil in hepatocyte and co-culture samples (FIGS. 5A and 5B). Further, absorption studies with empty devices exposed to these drugs showed no appreciable decrease up to 24 hours, excluding any artifacts due to PDMS.

The temporal profile (FIG. 4B) of 5-FU suggests that relatively quick turn over rate of 5-FU may prevent it from reaching toxic level in macro-scale cultures with high media volume. This is supported by results whereby MCF-7 toxicity was observed in micro-scale cultures but not in macro-scale cultures. Further, while the process of metabolism of 5-FU into subsequent secondary and tertiary metabolites is kinetic, the use of microfluidic models provides a unique opportunity to understand the mechanisms of the process, which has not been shown so far.

In summary, a versatile two-chamber microfluidic device that captures metabolic functions of cells, e.g., hepatocytes, has been developed, suggesting the importance of cell to media ratio in drug metabolism studies. The device demonstrates that microscale architecture recapitulates the metabolism of hepatocytes for drug screening. The present model provides a simple alternative for pro-drug metabolism studies.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining metabolism of a test compound, the method comprising:
(a) providing a device comprising:
a solid substrate;
a first member comprising a first inlet, a first chamber, and a first outlet;
a second member comprising a second inlet, a second chamber, and a second outlet; and
a liquid-permeable, cell-impermeable membrane;
wherein the first member is fixed to the substrate, and wherein the membrane is sandwiched and secured between the first and second members to provide a liquid-permeable, cell-impermeable barrier between the first and second chambers;
(b) introducing a suspension of first cells and the test compound into the first chamber of the device through the first inlet;
(c) introducing a suspension of second cells into the second chamber of the device through the second inlet;
(d) culturing the first and second cells by incubating the device; and
(e) determining viability of the second cells in the second chamber, thereby determining metabolism of the test compound by the first cells.

2. The method of claim 1, wherein the first cells are hepatocytes.

3. The method of claim 1, wherein the first cells are rat hepatocytes.

4. The method of claim 1, wherein the second cells are cancer cells, primary cells, or renal proximal tubule cells.

5. The method of claim 4, wherein the second cells are breast cancer cells.

6. The method of claim 1, wherein the test compound is tegafur, 4-ipomeanol, dacarbazine, trofosfamide, ifosfamide, or cyclophosphamide.

7. The method of claim 1, wherein the device is incubated at 37° C. at 5% CO₂.

8. The method of claim 1, wherein between 0.5 microliters and 20 microliters of the suspension of first cells is introduced into the first chamber of the device.

9. The method of claim 1, wherein between 0.5 microliters and 20 microliters of the suspension of second cells is introduced into the second chamber of the device.

10. The method of claim 1, wherein a ratio of volume of liquid in a chamber to the number of cells in the chamber ranges from 0.1 to 2.0 nanoliters per cell.

11. The method of claim 1, wherein the solid substrate is a glass slide.

12. The method of claim 1, wherein the first and second members comprise polydimethylsiloxane (PDMS), polystyrene, or cyclic olefin copolymer (COC).

13. The method of claim 1, wherein the membrane comprises polyethylene terephthalate (PET), polycarbonate, nylon, Mylar, stainless steel, wire mesh, aluminum, synthetic mesh, spectra, Kevlar, plastic, or paper.

14. The method of claim 1, wherein the membrane is secured to the first and second member by polydimethylsiloxane (PDMS).

15. A device comprising:
a solid substrate;
a first member comprising a first inlet, a first chamber, and a first outlet;
a second member comprising a second inlet, a second chamber, and a second outlet; and
a liquid-permeable, cell-impermeable membrane;
wherein the first member is fixed to the substrate, and wherein the membrane is sandwiched and secured between the first and second members to provide a liquid-permeable, cell-impermeable barrier between the first and second chambers.

16. The device of claim 15, wherein the solid substrate is a glass slide.

17. The device of claim 15, wherein the first and second members comprise polydimethylsiloxane (PDMS), polystyrene, or cyclic olefin copolymer (COC).

18. The device claim 15, wherein the membrane comprises polyethylene terephthalate (PET), polycarbonate, nylon, Mylar, stainless steel, wire mesh, aluminum, synthetic mesh, spectra, Kevlar, plastic, or paper.

19. The device of claim 15, wherein the membrane is secured to the first and second member by polydimethylsiloxane (PDMS).
